(19) United States
(12) Patent Application Publication
(10) Pub. No.: US 2016/0000054 A1
(43) Pub. Date: Jan. 7, 2016

(54) HUMAN-DERIVED MUTANTS OF THE dSOD1 GENE IN DROSOPHILA AND METHODS OF MAKING AND USING

(71) Applicant: Brown University Research Foundation, Providence, RI (US)

(72) Inventor: Robert Reenan, Rumford, RI (US)

(73) Assignee: Brown University Research Foundation, Providence, RI (US)

(21) Appl. No.: 14/741,534

(22) Filed: Jun. 17, 2015

Related U.S. Application Data
(60) Provisional application No. 62/013,779, filed on Jun. 18, 2014.

(51) Int. Cl. A01K 67/027; G01N 33/50; C12N 9/02; C12Q 1/68

(52) U.S. Cl. CPC A01K 67/0278; C12Q 1/6883; G01N 33/5085; C12N 9/0089; A01K 2207/15; A01K 2217/072; A01K 2227/706; A01K 2267/0318; C12Q 2600/124; C12Y 115/01001; C12N 2015/8536

(57) ABSTRACT

Genetic models of amyotrophic lateral sclerosis (ALS) are described, which can be used to identify novel treatments of ALS and therapeutic targets. Methods for making and using human-derived mutants of the Drosophila dSOD1 gene that model familial ALS are provided. Methods of identifying therapeutic ALS gene targets also are provided.

### RELATED APPLICATION

This application claims the benefit under 35 U.S.C. 119(e) of U.S. provisional application Ser. No. 62/013,779, filed Jun. 18, 2014, the entire contents of which are incorporated by reference herein.

### FIELD OF THE INVENTION

Provided herein are genetic models of amyotrophic lateral sclerosis (ALS), which can be used to identify novel treatments of ALS and therapeutic targets.

### BACKGROUND OF THE INVENTION

One common cause of familial ALS was discovered more than two decades ago, namely, mutations in Superoxide Dismutase 1 gene. Despite this advance knowledge, there are no therapies that dramatically alter the outcomes for a patient diagnosed with ALS. Most cases of familial ALS, referred to herein as fALS, are caused by mutations in the enzyme superoxide dismutase 1 (SOD1). Even though fALS accounts for only 10% of all ALS cases, 20% of fALS results from dominantly-inherited mutations in one gene, SOD1(1). In addition, other rarer genetic forms of ALS have been mapped to TDP-43 and FUS/TLS (DNA/RNA-binding proteins) and optineurin (involved in NF-kB signaling), all proteins that can be found in SOD1-positive inclusions (2-3) but play, as of yet, undefined roles in causing ALS. Considering SOD1 in transgenic mouse models, there are still a number of candidate cellular routes by which mutant SOD1 causes cell death. ALS-linked SOD1 mutant protein has been shown to selectively aggregate and localize to mitochondria of motor neurons, perturbing function (4). Another study implicates intercellular trafficking and protein secretory defects in cells expressing mutant SOD1 (5). Along these lines, motor-neuron specific ER-stress responses to mutant SOD1 have been suggested as a key mediator of neuronal toxicity (6). ALS-mutant SOD protein has also been suggested to indirectly perturb microtubule function and stability through several mechanisms (7-8). Lastly, SOD1 expressing motor neurons have been shown to display altered membrane biophysical properties and dendritic processing (9). Clearly, there are many cellular pathways affected by mutations in SOD1.

### SUMMARY OF THE INVENTION

Reverse genetics has been used to engineer Drosophila overexpressing wild-type human SOD1 (referred to herein as hSOD1) with various ALS-causing mutations. Such approaches have provided evidence for the importance of SOD1 activity for normal adult fly life span, and have shown that perturbing SOD1 activities can lead to neurological phenotypes. Nevertheless, there are clear drawbacks when using this heterologous overexpression approach in terms of assessing normal folding and activity of hSOD enzymes in Drosophila, and correlation of over-expression phenotypes to human disease-relevant symptoms. For instance, no studies have recapitulated substantial loss of motor control and motor neuron (MN) death by neuronal expression. Hence, there exists a need for the creation of accurate SOD1 mutant genetic models in Drosophila, which could be used to identify treatments and therapeutic targets for familial ALS involving hSOD1 mutations. Such a model should have the mutations engineered into the endogenous gene, which in the case of SOD1 is ubiquitous and highly expressed (˜1% total cell protein).

Notably, in biochemical, cell-based, and genetic systems, ALS mutations in SOD produce a wide range of effects, not always corroborative. The strongest approach possible would be to accurately model numerous ALS-causing mutations into an endogenous SOD gene, carefully characterize these models at molecular and cellular levels, and then perform forward genetics to find genetic modifiers. The method that provides as much accuracy as can be obtained is the use of homologous recombination. The power and pace of Drosophila can be used to generate models, and then proceed on to develop “genetic cures” based on those models to identify potential pathways that, when appropriately modified, forestall or eliminate neuronal cell death caused by ALS mutations.

Disclosed herein, is a model of ALS in the genetically tractable system, Drosophila melanogaster (fruit fly). Introducing human disease-causing mutations into the fly genome generates models that recapitulate the most important aspects of ALS, the lethal loss of motor control.

An allelic series of SOD1 mutations has been created that are increasingly deadly in the fly, with the G85R allele conferring unconditional lethality. Moreover, the lethal phase in flies is highly relevant to human ALS. For instance, flies carrying the dSod1-G85R allele all die at the end of metamorphosis, or after only a day or two of adult life, dependent upon gene dosage. The dying animals exhibit a characteristic lack of (or loss of) all motor activity, even while they are completely normal morphologically. Thus, while the endogenous dSOD1 gene is expressing the ALS mutant allele in all cells (like the human condition), the phenotypes observed are consistent with predominant effects on the nervous system, perhaps with MNs being most susceptible. Assessing animals earlier reveals that the larval neuromuscular junction shows dramatic changes in synapse size indicative of presynaptic deficits in motor neuron health.

Addressing this need, the following disclosure includes the use of homologous recombination (HR) to introduce human ALS-causing mutations into the endogenous fly SOD1 gene (referred to herein as dSOD1), generating the most faithful model of the human genetic architecture of ALS that is possible in Drosophila. This approach also has the benefit of simultaneously generating the matched genetic control animals.

To our knowledge, this is the most relevant model of ALS in any invertebrate genetic system. This model, due of the nature of the mutations and phenotypes, allows for the opportunity to identify pathways and proteins of potential therapeutic value in a way agnostic to current models of the etiology of ALS, and thus, may reveal novel new treatments and pathways involved in ALS progression.

Methods for making and using human-derived mutants of the Drosophila dSOD1 gene are provided. One aspect of the invention relates to a mutant Drosophila organism comprising a mutant dSOD1 gene that comprises at least one human-derived SOD1 mutation, wherein the mutant dSOD 1 gene replaces at least one of the copies of the endogenous dSOD1 gene. In one embodiment, the point mutation G85R, which replicates a known human hSOD1 mutation, is introduced into one the endogenous dSOD1 gene of a Drosophila organism by homologous recombination. Another aspect of the invention relates to a mutant Drosophila organism comprising a mutant dSOD1 gene that comprises a humanized dSOD1 gene, wherein the humanized dSOD1 gene replaces at least one of the copies of the endogenous dSOD1 gene. In one embodiment, the humanized dSOD1 gene is hSOD1. It should be appreciated that the humanized dSOD1 gene may be used to model any number of known hSOD1 mutations in a Drosophila organism.

Also provided herein are methods for making a dSOD1 mutant Drosophila organism comprising the steps of (a) generating a nucleic acid targeting vector comprising a dSOD1 gene with at least one human-derived mutation; (b) transforming a Drosophila organism with said targeting vector; and (c) identifying mutants wherein the dSOD1 gene with at least one human-derived mutation replaces at least one of the copies of the endogenous dSOD1 gene. In one embodiment, this method is used to produce a mutant Drosophila organism having one mutant H71Y dSOD1 gene in place of one of the endogenous dSOD1 genes. Additionally, methods of making a humanized dSOD1 mutant Drosophila organism are disclosed, comprising the steps of (a) generating a nucleic acid targeting vector comprising a humanized dSOD1 gene; (b) transforming a Drosophila organism with said targeting vector; and (c) identifying mutants wherein the humanized dSOD1 gene replaces at least one of the copies of the endogenous dSOD1 gene.

According to another aspect, methods of identifying therapeutic ALS gene targets are provided. The methods include the steps of (a) contacting a human-derived dSOD1 mutant Drosophila organism with an effective amount of mutagen; (b) generating progeny of said mutant organisms; (c) identifying progeny wherein the phenotype of the mutant organism is suppressed or enhanced; (d) sequencing the transcriptome of rescued progeny; and (e) identifying the mutant genes. In one embodiment, suppressor mutations are identified by transcriptome sequencing to reveal novel therapeutic ALS gene targets.

According to another aspect, methods of identifying a compound for treating an ALS patient in need of treatment are provided. The methods include the steps of (a) contacting a human-derived dSOD1 mutant Drosophila organism with a library of compounds; and (b) identifying compounds that suppress the phenotype of the mutant organism. In one embodiment the library of compounds refers to a small molecule library of compounds, while in other embodiments, the library of compounds are proteins or nucleic acids.

These and other aspects and embodiments of the invention are described further below.

### BRIEF DESCRIPTION OF THE FIGURES

FIG. 1 shows the generic ends-out homologous recombination in Drosophila. Panel A shows a pW25 P-element targeting vector (thick black line with inverted repeats represented as filled triangles) randomly inserted in the Drosophila genome (thin line). Within the transposable element are: homology arms 1 and 2 for the gene of interest (light gray) and the mW+ eye color gene. Direct repeats of FLP and CRE recombinase sites are shown as white and gray triangles, respectively. Vertical arrowhead is the SCEI restriction endonuclease cutting site. Mutation in arm 1 is depicted as an asterisk. Panel B is a schematic showing that induction of FLP recombinase (1) mobilizes the targeting construct as an episomal circle. Subsequent cutting by the SceI endonuclease (2) generates the linear recombinogenic intermediate. Panel C depicts homologous recombination occurring at the endogenous locus (solid light gray box) within regions that overlap arms 1 & 2. Panel D shows an endogenous locus with mutation and mW+ marker (usually in an intron). At this step, the mW+ can inactivate gene expression, preventing mutant expression. Panel E is a schematic showing that induction of CRE recombination (3) excises the mW+ as a circle, leaving behind a single loxP site gray triangle).

FIG. 2 shows Helicos deep sequencing data. A plot of tag-wise dispersion comparing dADAR+ and dADAR− animals can be found. Between the two backgrounds it can be found that ˜40 genes were upregulated >4-fold and ˜80 genes were downregulated >4-fold. Adjusted P-values are highly significant for these genes. Most importantly, many genes of interest for ALS research were detected robustly in our data set over a 500-fold expression range (e.g. SOD, Dorfin, TDP-43, Hsc70, cathepsin D, TNFa, laminin a-2, LYST, ter94, Tau, GstS1, Tor, Thor, and many Heat shock proteins). Thus, we are confident in our ability to detect changes in transcriptional profiles in our dSOD mutants, as well as other backgrounds.

FIG. 3 shows an amino acid sequence alignment of dSOD1 (SEQ ID NO: 2) (top sequence) with human SOD1 (SEQ ID NO: 3) (bottom sequence). The identical amino acids between the two sequences are shown between the dSOD1 and human SOD1 sequences (middle sequence). The dSOD1 protein is 60.6% identical to the human SOD1 protein.

### DETAILED DESCRIPTION OF THE INVENTION

Amyotrophic lateral sclerosis (ALS) is a devastating neurodegenerative disease that manifests itself as a progressive loss of motor neurons (MNs). An initial reduction in mobility is followed by paralysis with the most common cause of death a result of respiratory failure. One common cause of familial ALS was discovered more than two decades ago, namely, mutations in the Superoxide Dismutase 1 gene (SOD1). Despite this advance knowledge, there are no therapies that dramatically alter the outcomes for a patient diagnosed with ALS. Approximately 10% of cases have been associated with a genetic lesion (familial ALS) and 20% of these cases are linked to a mutation in the SOD1 gene. Such mutations are dominant and are thought to represent a gain of function for the mutant form of the SOD1 protein. ALS animal models developed to date have generated transgenic lines that overexpress mutant forms of SOD1. Hence, there is a need to better model the genetic, cellular, and systemic state of an ALS patient.

To address this need, described herein is the use of homologous recombination (HR) to knock-in specific genetic changes into the homologous Drosophila Sod1 gene, referred to herein as dSod1. Aspects of the disclosure relate to making and using Drosophila mutant organisms to identify novel gene targets and therapeutics. For example, Drosophila containing mutations in their dSod1gene may be used as a model system for identifying novel gene targets and therapeutics for familial amyotrophic lateral sclerosis (ALS).

For example, dSod1-ALS mutants generated by HR recapitulate the substantial loss of motor control and motor neuron (MN) function seen in human ALS. A series of ALS mutations of increasing severity were created, with the dSod1-G85R allele conferring an early-onset severe phenotype with <0.1% dSod1-G85R adults emerging from the pupal case at the end of metamorphosis (a feat requiring considerable muscle strength) and other alleles (H71Y and G51S) resulting in a later onset form of the disease that leads to a later lethal phase in adult life (˜two weeks). In mutant animals earlier in development the larval neuromuscular junction shows dramatic changes in synapse function indicative of presynaptic deficits or upstream motor neuron defects. In addition, there are changes that indicate disease progression may be associated with increasingly severe impairment of neuronal function. These phenotypes may be used to identify biomarkers of disease as well as factors that modulate disease progression. As one example, this model has been used to demonstrate that activation of the BMP signaling pathway can suppress early defects in motor function and appears to extend both the healthspan and lifespan of dSod1-G85R mutants.

It is believed that this is the most genetically accurate model of ALS in an experimental system and is useful to study the nature of the effect of SOD1 mutations on nervous system function, as well as determine their genome-wide transcriptomic effects, first, as a molecular diagnostic signature of the disease and its mechanistic underpinnings, and second, as a means to identify therapeutics, genes or pathways that will provide an entry point for therapeutic development. For example, this model was used to discover that bone morphogenic protein (BMP) signaling can rescue mutant SOD1-induced ALS phenotypes. This not only reveals a potential therapy, but provides a method to compare ALSdSod1 mutant and BMP-rescued animals at both the organismal and transcriptomic level, a powerful method for pinpointing pathways capable of reversing MN dysfunction and death in ALS. Using this model, one may characterize the progressive nature of an allelic series of ALS mutants with respect to behavioral, immunocytological, and cellular physiologic changes, correlate these changes with transcriptomic changes during the same time course of disease progression and to also characterize the nature of BMP signaling rescue in terms of the same phenotypic changes seen during disease progression as well as analyze rescued animals transcriptomically to identify pathways or processes that are altered in the disease animals upon rescue, in order to identify potentially new insights into therapeutic approaches.

Drosophila SOD1 (dSOD1) Mutant Models
Replacing the Endogenous dSOD1 Gene

Aspects of the disclosure relate to dSOD1 mutants and uses thereof. For example, a mutation, or mutations, may be introduced into the endogenous dSOD1 gene (NCBI GENE ID #39251, and as shown below) of Drosophila by homologous recombination.

>gi|442631645|ref|NM_057387.5|Drosophila
melanogaster superoxide dismutase (Sod),
transcript variant A, mRNA
(SEQ ID NO: 1)
CCGCATGTATTTCTAAGCTGCTCTGCTACGGTCACACCATAGAAGAT
ACCTGGAAAGTTCTCAACTTTTTTCGTTTTGATAAATTGATTAATTC
ATTCGAAATGGTGGTTAAAGCTGTCTGCGTAATTAACGGCGATGCCA
AGGGCACGGTTTTCTTCGAACAGGAGAGCAGCGGTACGCCCGTGAAG
GTCTCCGGTGAGGTGTGCGGCCTGGCCAAGGGTCTGCACGGATTCCA
CGTGCACGAGTTCGGTGACAACACCAATGGCTGCATGTCGTCCGGAC
CGCACTTCAATCCGTATGGCAAGGAGCATGGCGCTCCCGTCGACGAG
AATCGTCACCTGGGCGATCTGGGCAACATTGAGGCCACCGGCGACTG
CCCCACCAAGGTCAACATCACCGACTCCAAGATTACGCTCTTCGGCG
CCGACAGCATCATCGGACGCACCGTTGTCGTGCACGCCGATGCCGAT
GATCTTGGCCAGGGTGGACACGAGCTGAGCAAGTCAACGGGCAACGC
TGGTGCCCGCATCGGGTGCGGCGTTATTGGCATTGCCAAGGTCTAAG
CGATAATCTATTCCGATGTCGGCCACTGTGCTGATCTACTCTATTTA
GCACTACCCACTGGAGATATACAAACGATATACATACTTCTAAACAT
AAATACATAGCCTGTGGTCTGTTAGTTGATACGCAACCTTTGAGGTT
CAATAAATTGGTGTTTTGAAATTGCCCCAT

Herein, homologous recombination refers to a type of genetic recombination in which nucleotide sequences are exchanged between two similar or identical molecules (homologous sequences) of DNA. In some embodiments homologous recombination is performed with a nucleic acid targeting vector. Herein, a nucleic acid targeting vector refers to a nucleic acid molecule that integrates into a specific genomic location. In some embodiments, the nucleic acid targeting vector comprises sequence homology to the dSOD1 locus. In some embodiments the targeting vector is linearized by a linearizing endonuclease. It should be appreciated that recent technical advances have made it feasible to target and replace endogenous sequences in the fly genome using homologous recombination (Rong and Golic, 2000; Rong et al., 2002; Staber et al., 2011). In some embodiments homologous recombination replaces at least one endogenous dSOD1 allele with a mutant dSOD1 allele. In some embodiments the homologous recombination used is ends-out homologous recombination. In some embodiments, homologous recombination is used for gene targeting. Herein, gene targeting refers to a genetic technique that uses homologous recombination to replace an endogenous gene. In some embodiments, homologous recombination is used to delete a gene, remove exons, add a gene, introduce point mutations or introduce multiple mutations into the genome. In some embodiments, gene targeting can be permanent or conditional. In some embodiments, the term conditional may refer to a specific time during development or life of the organism, limitation to a specific tissue or following exposure to an inducing agent. For example, gene targeting requires the creation of a specific targeting vector for each gene of interest. Notably, it can be used for any gene, regardless of transcriptional activity or gene size.

Generating Human-Derived dSOD1 Mutants

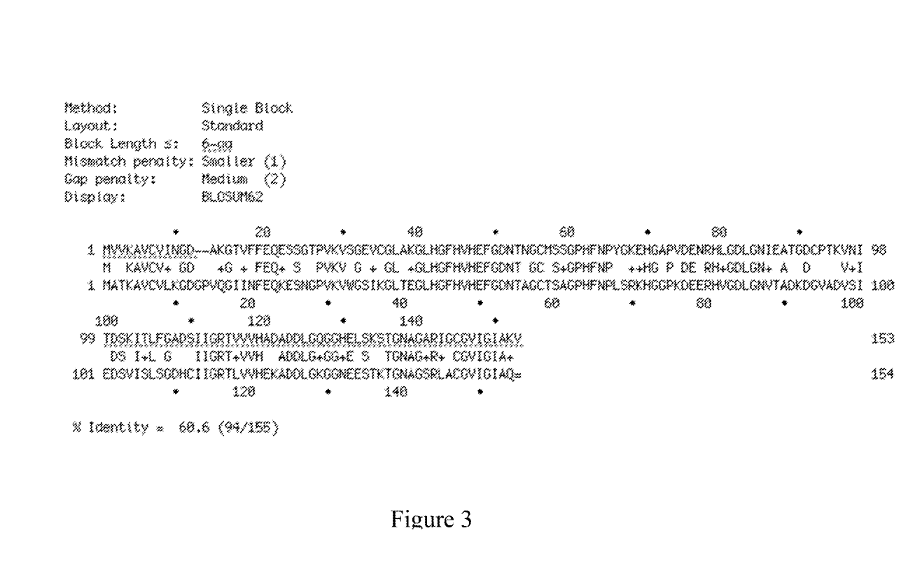

Taking into consideration the hSOD1 protein itself, and the nature of the mutations, it is known that many mutations (>100) occur in such a small protein (˜150 amino acids) and while this has been known for 17 years (13), treatments aimed at intervening directly in mutant hSOD1's effects have remained elusive. Aspects of the disclosure relate to dSOD1 mutants that model the repertoire of human hSOD1 mutations. As one example, one copy of the dSOD1 gene is replaced with a mutant copy of dSOD1 gene, by homologous recombination, having at least one human-derived dSOD1 mutation. Herein, a human-derived dSOD1 mutation refers to any human hSOD1 mutation that can be identically mutated on the homologous dSOD1 gene. It should be appreciated that the hSOD1 and dSOD1 proteins share 60.6% identity, having 61 amino acid changes (FIG. 3), and therefore some human-derived hSOD1 mutations cannot be modeled in the endogenous Drosophila dSOD1 gene. The dSOD1 gene encodes the protein having the amino acid sequence:

(SEQ ID NO: 2)
MVVKAVCVINGDAKGTVFFEQESSGTPVKVSGEVCGLAKGLHGFHVHE
FGDNTNGCMSSGPHFNPYGKEHGAPVDENRHLGDLGNIEATGDCPTKV
NITDSKITLFGADSIIGRTVVVHADADDLGQGGHELSKSTGNAGARIG
CGVIGIAKV.

In some embodiments, the human-derived dSOD1 mutation is a deletion, an insertion, a point mutation or multiple mutations. In some embodiments, the human-derived dSOD1 mutation is A4S, A4V, G37R, H48R, H71Y, G85R, R115G, D124G, G141E, G147R or C6S. It should be appreciated that the human-derived dSOD1 mutation may be any single hSOD1 mutation or any combination of more than one hSOD1 mutations.

Humanized dSOD1 Gene

In some embodiments, at least one endogenous dSOD1 gene is replaced with a humanized dSOD1 gene. Herein, a humanized dSOD1 gene refers to a gene in which the endogenous SOD1 gene has been engineered to encode the entire homologous human SOD1 gene in the context of the Drosophila organism. Alternatively, introducing at least one point mutation into the endogenous dSOD1 gene that changes a non-identical amino acid (i.e., non-identical between dSOD1 and hSOD1) to the identical amino acid occurring in hSOD1 constitutes a partially humanized dSOD1 gene. Thus the partial or complete humanization of the dSOD1 gene involves changing up to 61 non-human amino acid residues in dSOD1 to the human SOD1 amino acid residues. Doing so permits modeling the human-derived hSOD1 mutations that cannot be modeled in the endogenous Drosophila dSOD1 gene.

The partial or complete humanization of the dSOD1 gene can be accomplished by standard DNA synthetic techniques to generate the humanized-dSOD1 in the context of the endogenous and homologous dSOD1 flanking sequences. Using standard HR techniques, such a construct then is incorporated into the Drosophila genome, in which now, the partially or completely humanized dSOD1 locus produces a protein that is partially or fully identical in primary amino acid sequence to the human SOD1.

In some embodiments, the partially humanized dSOD1 gene encodes a protein having at least 60%, 65%, 70%, 75%, 80%, 85%, 90%, 95%, or 99% sequence identity to the hSOD1 protein. In some embodiments, the partially humanized dSOD1 gene encodes a protein having between 1 and 60 amino acid substitutions. It should be appreciated that any manner of the human-derived mutations may be introduced into the humanized or partially humanized dSOD1 gene. For example, the D90A mutation, which has been studied in a mouse over-expression model, is not conserved in fly but may be modeled using a humanized or partially humanized dSOD1 gene.

### SOD1 Mutant Models

It should be appreciated that attempts to delineate the cellular pathways involved in ALS have used mouse models and even patient samples to perform genome-wide expression profiling. Two different studies using SAGE and cDNA macroarray analyses of ALS-transgenic mice (G93A) revealed numerous changes in gene expression between presymptomatic or symptomatic animals and controls (10-11). Genes acting in a wide variety of pathways were up- and down-regulated including apoptosis, oxidative stress, protein turnover and axonal transport. One of these studies went so far as to confirm similar expression changes to the mouse model in ALS patient samples (11). Another study of the same transgenic mouse model used expression arrays to identify co-stimulatory pathway components and validated the use of anti-CD40L as a potential palliative treatment (12). These studies have provided a more clear picture of the cellular and genomic responses to mutant SOD proteins. However, less clear is how powerful and effective interventions can be achieved utilizing these methods, which reveal the multivariate, system-wide nature of cellular perturbations in ALS. It is difficult to detect which alterations might be most important for the progression and severity of disease.

While is it not controversial to state that ALS mutations in SOD1 cause protein aggregation and inclusion formation, divergent interpretations of the molecular nature of SOD1 aggregation have been proposed. For example, one study has asserted that SOD1 aggregates are composed of unmodified SOD1 protein, alone, with no other associates (14) while a different study convincingly demonstrates that there is a protein modification that serves as a “molecular signature” of both fALS and sporadic ALS (15). Convincing evidence for a modification that facilitates oligomerization is the formation of intermolecular disulfide bonds between aberrantly folded mutant SOD1 protomers (16-17). These studies showed that in addition to the normal intramolecular disulfide bond formed in SOD1 (C57-C146), two free Cys residues (C6 and C111) play a substantial role in the in vivo aggregation and toxicity of mutant SOD1. In fact, other studies indicate that the misfolded SOD1 in its unmetallated state, early in protein folding and maturation, is prone to intermolecular disulfide bond formation (18). Thus, there is substantial data to indicate that protein folding and intermolecular covalent modifications are crucial for the pathogenicity of ALS mutant SOD1.

Accepting that SOD1 is toxic as an aggregate, regardless of its pathogenic target, one is faced with the additional insult to proteostasis and removal of toxic aggregates. Mutant SOD1 has been shown to form polyubiquitin-dependent inclusions (19). A E3-ubiquitin ligase capable of ubiquitylating SOD1, Dorfin, was shown to localize to inclusion bodies of fALS and sporadic ALS patients (20). Ubiquitination of mutant SOD by Dorfin was also shown in several studies to alleviate SOD1 toxicity in neuronal cell cultures overexpressing Dorfin, where mutant SOD1 showed reduced localization to mitochondria (20-21). Additionally, overexpression of Dorfin substantially forestalled the onset of neurological deficits and death in the G93A transgenic mouse model, indicating a therapeutic effect of Dorfin on disease progression (22). In addition to Dorfin, Valosin-containing protein (VCP) has been shown to functionally interact with Dorfin and regulates the ubiquitylation of SOD1 (23). This is an intriguing connection since VCP has been shown to be mutated in IBMPFD, a dementia, and to be localized to ALS and PD inclusions.

Biochemical isolation of aggregated SOD1 protein from transgenic mice has resulted in some compelling evidence for pathogenic protein associations. Two different studies revealed that mutant (but not wild type) SOD1 protein associated strongly with the HSC70 protein, a cytosolic chaperone (24-25). Other proteins associated with ER-stress and proteostasis, as well as cytoskeleton were also identified. Constitutively expressed HSC70 proteins, related to the stress-induced HSP70s, have been shown to be essential for normal neurotransmitter exocytosis (26), as well as the normal functional expression of certain ion channels at the cell surface (27). Thus, it seems likely that aggregated mutant SOD1 proteins are associated dynamically with, and are remodeled by, numerous chaperones and components of protein degradation pathways.

Recent data suggest that transgenic expression of SOD1 containing ALS mutations in mouse neurons alone is sufficient to produce ALS-like symptoms in animals (28). The SOD1 protein is highly conserved. It would be tempting to address the toxicity of ALS mutant SOD1 in a facile and inexpensive system which could potentially reproduce the cellular mechanisms of neuronal cell death in response to mutant SOD1. Drosophila is a very powerful genetic system with the potential for very effective forward genetic screens for phenotypic modifiers. Flies lacking dSOD are viable, but short lived. One study used transgenic expression of human SOD1 (hSOD) and various ALS mutant forms in a dSOD-genetic background (29). While the study was able to rescue some longevity defects with specific hSOD constructs, the data was confounded by lack of expression of hSOD activity and the intrinsic defects present in the dSOD-background. A different study used targeted expression in Drosophila motor neurons (30). Here, aggregation of hSOD1-G85R was shown to occur in an age-dependent manner, decreased climbing ability with age, and age-dependent changes in neuronal electrophysiology. The same observations were seen with engineered expression of wild-type hSOD. Yet, similar experiments using transgenic expression of hSOD1-G85R in the nematode, C. elegans, produced neuronal inclusions and locomotor defects at all ages tested, only for ALS-mutant hSOD (31). Wild type hSOD expression had no effect.

### Need for More Accurate SOD1 Mutant Models

Clearly, in biochemical, cell-based, and genetic systems, ALS mutations in SOD produce a wide range of effects, that are not always corroborative. The strongest approach possible is to accurately model numerous ALS-causing mutations into an endogenous SOD gene, carefully characterize these models at molecular and cellular levels, and then perform forward genetics to find genetic modifiers. The method of generating human-derived and humanized dSOD1 mutants by HR, described herein, provides as much accuracy as can be obtained in this model. Studies suggest that SOD1-based ALS may proceed through non-cell-autonomous mechanisms in which other cells, which are also mutated for SOD1, are causative in the death of motor neurons, rather than motor neurons themselves. This makes a compelling argument for an accurate genetic model, as overexpression in motor neurons alone will almost certainly not recapitulate the molecular etiology of ALS. The power and pace of this Drosophila model can be used to develop “genetic cures” based on those models to identify potential pathways that, when appropriately modified, forestall or eliminate neuronal cell death caused by ALS mutations. This modeling of specific disease causing SOD1 mutations into the fly genome has the potential to provide a rapid and low-cost platform for studying the cellular mechanisms of heritable human diseases. In some embodiments, human-derived dSOD1 mutants or humanized dSOD1 mutants can be used in combination with forward genetic screens to identify suppressor and/or enhancer mutations. In other embodiments, human-derived dSOD1 mutants or humanized dSOD1 mutants can be used in compound screens to identify small molecule, protein or nucleic acid based therapeutics for fALS involving SOD1 mutations.

### Identifying Novel Therapies and Therapeutic Gene Targets for Familial ALS

Aspects of the disclosure relate to the use of genetic screens to identify novel therapeutic gene targets and pathways to treat familial ALS. Herein, a genetic screen refers to an experimental technique used to identify and select for individuals who possess a phenotype of interest in a mutagenized population. For example, a Drosophila organism having a human-derived mutation in dSOD1, where at least one endogenous dSOD1 gene is replaced by the human-derived mutant dSOD1 gene, is contacted with a mutagen to produce Drosophila mutant organisms that modify the phenotype of the human-derived mutant dSOD1 Drosophila organism. In some embodiments, the genetic screen is a forward genetic screen. In some embodiments, the human-derived dSOD1 mutation is A4S, A4V, G37R, H48R, H71Y, G85R, R115G, D124G, G141E, G147R or C6S. In some embodiments, the humanized dSOD1 mutant phenotype is a lethal phenotype, a larval phenotype, or an adult phenotype. In some embodiments the humanized dSOD1 mutant phenotype is a neuromuscular phenotype. It should be appreciated, that human-derived dSOD1 mutant Drosophila organisms may display phenotypes of various types and degrees of severity.

In some embodiments the mutagen is EMS. In some embodiments, enhancer mutations of human-derived dSOD1 mutant Drosophila organisms are identified. Herein, enhancer mutations refer to mutations that exacerbate, or enhance a phenotype of interest in an already mutant individual. This screen can identify additional genes or gene mutations that play a role in that biological or physiological process. In some embodiments, suppressor mutations of human-derived dSOD1 mutant Drosophila organisms are identified. Herein, suppressor mutations refer to mutations that alleviate or revert the phenotype of the original mutation. Suppressor mutations may be second mutations at the site on the chromosome distinct from the original mutation, which suppresses the phenotype of the original mutation. If the mutation is in the same gene as the original mutation it is known as intragenic suppression, whereas a mutation located in a different gene is known as extragenic suppression or intergenic suppression.

It should be appreciated that mutations including but not limited to enhancer and suppressor mutations may identify novel therapeutic ALS gene targets. In some embodiments, therapeutic gene targets are genes that, when mutated, enhance or suppress the phenotype of a dSOD1 mutant Drosophila organism. It should be appreciated that enhancer or suppressor mutations may be identified by a number of known techniques in the art. In some embodiments, therapeutic ALS gene targets are identified by genetic mapping. In some embodiments, therapeutic ALS gene targets are identified by positional cloning. In some embodiments, therapeutic ALS gene targets are identified by genome sequencing. In some embodiments, therapeutic ALS gene targets are identified by sequencing the transcriptome.

While genetic models of ALS have attempted to understand the etiology of motor neuron cell death seen in ALS-causing SOD mutations, none of the systems is sufficient for forward genetics since overexpression systems do not mimic the genetic architecture of human ALS. Forward genetic suppressor screens may hold the key to rapid identification of new avenues to therapeutic intervention by letting a valid model system answer the question, “What can restore the system to normalcy?” Disclosed herein is a knock-in model of human SOD-based ALS and methods for making and using. One may use the power of Drosophila forward genetics to identify, in an unbiased fashion, those pathways that when mutated can forestall, alleviate, or “cure” the disease symptoms (lack of motor activity and neuronal death) in the disclosed model system. Drosophila has a rich and successful history of forward genetics to probe pathways and processes using genetic enhancement and suppression. The most significant results of this approach would be to identify such pathways or processes that could point the way to novel therapeutic interventions in as short a time as possible. The disclosed approach solves an important problem in the field, as of the nature of the mutations and phenotypes, disclosed herein, present a rare opportunity to identify pathways and proteins of potential therapeutic value in a way agnostic to current models of the etiology of ALS, and thus, may reveal novel new pathways involved in ALS progression.

### EXAMPLES

In order that the invention described herein may be more fully understood, the following examples are set forth. The examples described in this application are offered to illustrate the mutants, and methods of making and using provided herein and are not to be construed in any way as limiting their scope.

### Example 1

### Methods

### Genome-Wide Transcriptional Profiling of ALS Mutants.

Deep sequencing via RNA-seq generates rapid and accurate expression profiling. The extent and complexity of eukaryotic transcriptomes have been redefined by the precise determination of transcript levels (and their isoforms) achieved through RNA-seq over traditional hybridization-based methods. This method was used with defined mutants that affect nervous system function and integrity (e.g. dADAR mutants) and have generated high-quality data of a type only obtained via single molecule sequencing where no amplification steps are involved. One may apply this methodology to the analysis of ALS-causing SOD mutations in Drosophila, under the premise that among the altered transcriptional states observed, one may identify perturbed pathways that are amenable to experimental or therapeutic intervention.

Poly(A)+ RNA is isolated from total RNA of female heads and thoraces of wild-type controls and all generated dSOD mutants. Samples are processed from both heterozyogotes over the wild-type control allele and homozygotes. In addition, samples are processed from cross-sectional aged populations at days 1, 10 and 40. Briefly, RNA is fragmented, and copied into complementary DNA (cDNA) (47). Fragments in the 200-300 bp range are isolated and prepared according to the manufacturer's protocol for the Paired-end Genomic DNA library (Helicos). Based upon experience, three biological replicates are adequate for assessing modest changes in gene expression with extreme confidence. It is known that technical replicates result in highly correlated data sets with miniscule differences.

Sequences are mapped to the Drosophila genome by using the ultra-fast short read alignment Bowtie program. One may use additional methods to quantitate unannotated or non-coding RNAs (44). Number of sequence reads is proportional to the levels of mRNA per specific gene. Gene expression is normalized by calculating the reads per kilobase of gene model per million mapped reads (RPKM). The fold change between the mutant flies and controls is calculated for those genes with an RPKM greater or equal to 2.0. Database for Annotation, Visualization and Integrated Discovery (DAVID, http://david.abcc.ncifcrf.gov) is a bioinformatic resource that will be used for systematic and integrative analysis, gene and functional classification (Huang et al. 2008). Bioinformatics tools generated by SLI specifically for analysis of single molecule sequencing data are used. Pathway analysis and identification of networks associated with dSOD mutant flies is done by Gene Ontology (GO), the KEGG and Pfam databases. Highly significant and interesting candidates are validated using real-time PCR.

### Generate and Identify Genetic Suppressor Mutations Capable of Alleviating the Deleterious Effects of ALS-Causing SOD Mutations.

As certain SOD mutations are likely to have serious behavioral/neurological consequences for affected animals, there is an interest in using the unbiased power of Drosophila forward genetics to obtain genetic suppressors of “disease”. First, a SOD residue that has been shown to play a role in disulfide-mediated aggregation, C6, is mutated. Mutation of this residue (C6S) was shown to dramatically reduce the amount of aggregation and toxicity of ALS-causing hSOD mutations. Since this residue is conserved in dSOD, we have generated a targeting vector to make C6S by HR. Since the C6S mutation is in arm1 of the targeting construct, one can easily combine it with any mutation introduced in arm2 (G37R, H48R, H71Y, G85R, R115G, D124G, G141E, G147R). In this way, one can determine proximally, in vivo, if reducing the ability of ALS-causing mutant SOD protein to form inter-protomer disulfide bonds affects disease phenotype and progression. While this method does not identify new protein suppressor, it serves as a rational proof-of-concept that suppression of disease phenotype through a mechanism (aggregation) can be efficacious.

A second existing “candidate” mechanism for assessing genetic suppression is the use of the C522T mutation in ter94. This mutation was shown to convey substantial oxidation resistance to the VCP protein in mammalian cells, and was shown to confer actual organismal oxidative stress resistance in S. cerevisiae. The mutation comparable to C522T was introduced into ter94, and it confers substantial resistance to H₂O₂ stress. Since VCP plays a role (with Dorfin) in ubiquitylating mutant SOD, important interactions between the C522T mutant and ALS causing SOD mutants in Drosophila are expected, validating VCP as a potential therapeutic intervention in ALS.

Genetic suppressors that reverse the effects (e.g. decreased lifespan, locomotor uncoordination) of ALS mutations in flies may identify genes whose human counterparts could be involved in the etiology of ALS. Mutageneses are performed on the most severe and disease-relevant dSOD mutants, including A4S, A4V, G37R, H48R, H71Y, G85R, R115G, D124G, G141E, G147R and C6S to select for suppressor mutations. This approach, using HR, provides simply the best genetic backgrounds in which to select for suppressor mutations. Once obtained, suppressor mutations are mapped to a chromosome by standard fly genetics and tested for suppression of other dSOD alleles. Suppressor mutations that stand up to initial genetic validation are identified by using NEXGEN sequencing to narrow down suppressor mutants rapidly, a technique that has been previously shown to be effective. In particular, since the ALS-suppressors reverse the effects of human mutations in animals and potentially block or forestall neuronal death, such ALS-suppressors point to novel and unexpected avenues for interventions that could block disease early in its genesis.

### Example 2

### Ends-Out Homologous Recombination in Drosophila

Drosophila melanogaster were used in the production of exacting models of human inherited disorders using the targeted method of ends-out homologous recombination (HR) (32). While HR has been used for specific projects in the field, it has not been utilized to this end. Instead, the construction of models of human disease in Drosophila has relied on more traditional methods. The most commonly used model is the Ga14 binary expression system which harnesses the yeast activator, Ga14, to drive expression of transgenes engineered with repeats of an upstream-activating sequence (UAS) near a gene-of-interest. Thus, flies expressing Ga14 under control of a tissue-specific promoter can be crossed to flies containing a UAS-gene construct to achieve targeted expression. This approach has been successfully employed to drive expression of a number of mutant proteins, including hSOD1, creating Drosophila disease models (29-30, 33-35). The binary system is also useful for driving expression of RNAi knockdown constructs to create phenocopies of protein hypomorph mutations. However, there are methodological challenges accompanying this approach, particularly for human diseases with complex etiologies and cell specific dominant expression including, (i) balancing expression levels (ii) determining the appropriate genetic control and, (iii) assessing the relevance of the resulting phenotype.

Protocols have been established that have made HR both reliable and efficient in the lab (36), including creating standardized cloning procedures and an “assembly-line” approach to vector construction. Construction of a targeting vector typically requires 1-3 weeks. A secondary concern has been a perceived low frequency and fidelity of targeting (37), which was not observed in this case. Targeted alleles can usually be found in an afternoon of screening by one researcher.

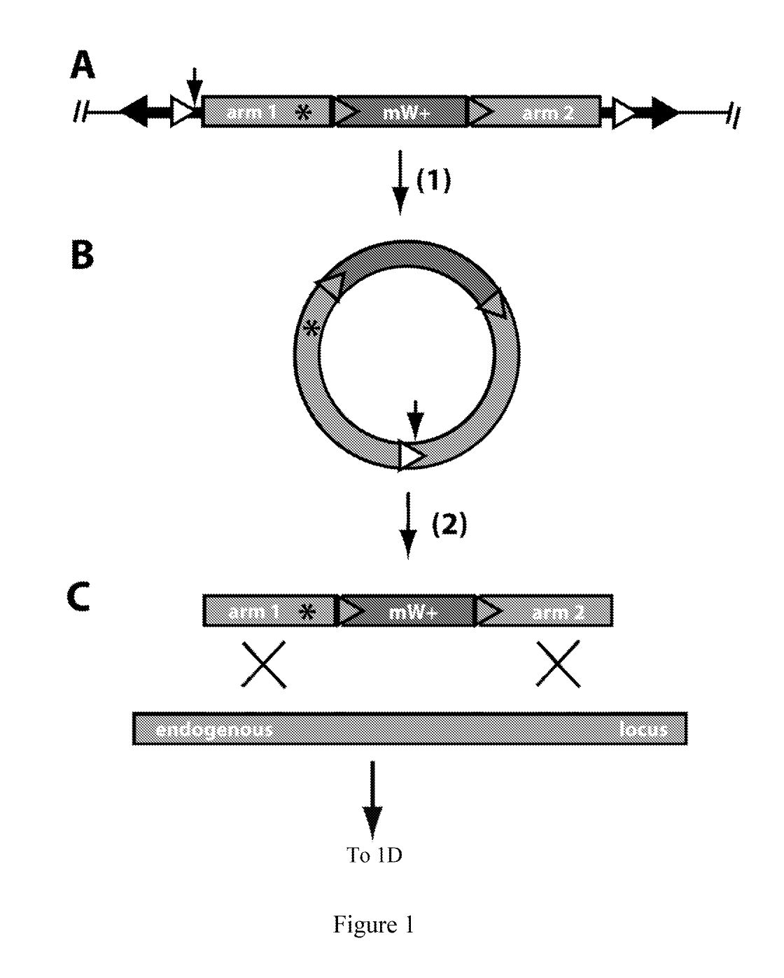

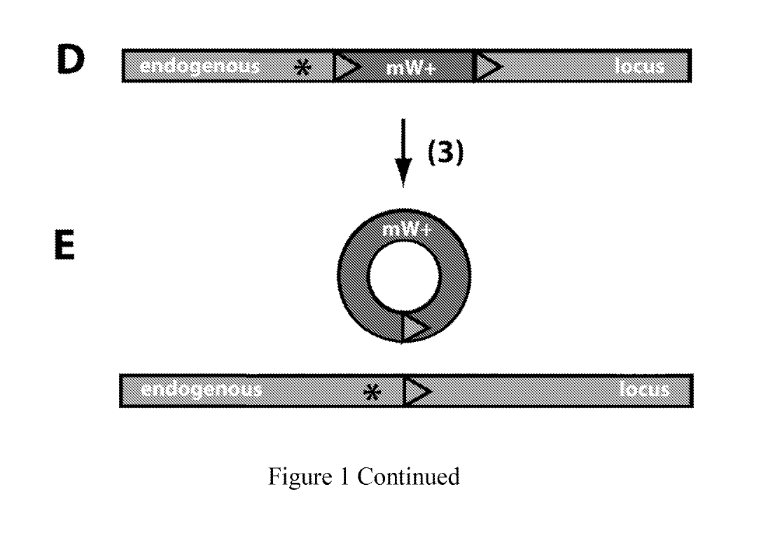

The HR strategy has several advantages for reading the complex genotype-to-phenotype mapping of human disease. First, in the course of performing HR, control targeted lines without a mutation are always generated. Such wild type control stocks (and subsequent mutant versions) are then backcrossed into a common genetic background. This approach allows the generation of control and mutant lines for direct comparison where all endogenous gene regulation remains intact. Second, an intermediate of the HR process is the generation of a “marked” allele containing a large insertion of the mini-white+ (mW+) eye-color gene (FIG. 1, Panels A-E).

Flanking the mW+ insertion are CRE recombinase sites that allow for subsequent removal. The insertion of mW+ is capable of generating a null-mutant phenotype for some genes (FIG. 1, Panel D). Thus, the technology exists to “turn on” a potentially highly deleterious dominant gain-of-function mutant protein in a cell-specific manner by targeted expression of the CRE recombinase using the Ga14 binary expression system. Another clear advantage to Drosophila HR is that, technically, generation of the initial wild type targeting vector is the most labor-intensive step of HR. Once made, subsequent generation of multiple mutant targeting constructs from the wild type is straightforward through the use of rapid and high fidelity site-directed mutagenesis techniques. Coupled with the ease of HR in Drosophila, the stage is set for the generation of allelic series of disease-causing mutations a powerful tool in correlating genotypes to phenotypes. Moreover, because the targeting scheme involves two separate targeting arms (FIG. 1, Panels A-C), two mutations can be combined in the same targeting construct. Thus, the potential to assess intragenic mutant interactions exists. Such combinations could reveal genetic interactions while probing structure/function relationships, in vivo, in a disease context.

Briefly, the procedure for the generation of a homologous recombinant is depicted schematically in FIG. 1, Panels A-E. A targeting vector (pW25) is constructed with two homology arms, each capable of containing one or more mutations. The construct is designed so that the selectable marker (mW+), flanked by CRE recombinase sites, is contained within intronic or other non-coding, non-conserved sequences of the final targeting event. Because 12 species of Drosophila have been sequenced and are available as a community resource, robust sequence comparisons can be performed to identify non-conserved regions for vector design.

Targeting proceeds through a series of genetic crosses to generate a recombinogenic intermediate (FIG. 1, Panels B and C). After targeting is confirmed for multiple independent events/vector (>3), a final reduction step with CRE recombinase leaves behind a single loxP site. The remaining loxP site is benign to gene expression. In fact, the engineered loxP site can be used to efficiently induce recombination between two alleles, which is a useful trick for genetically shuffling various dSOD alleles.

### Example 3

### RNA-Seq and Transcriptomic Analysis

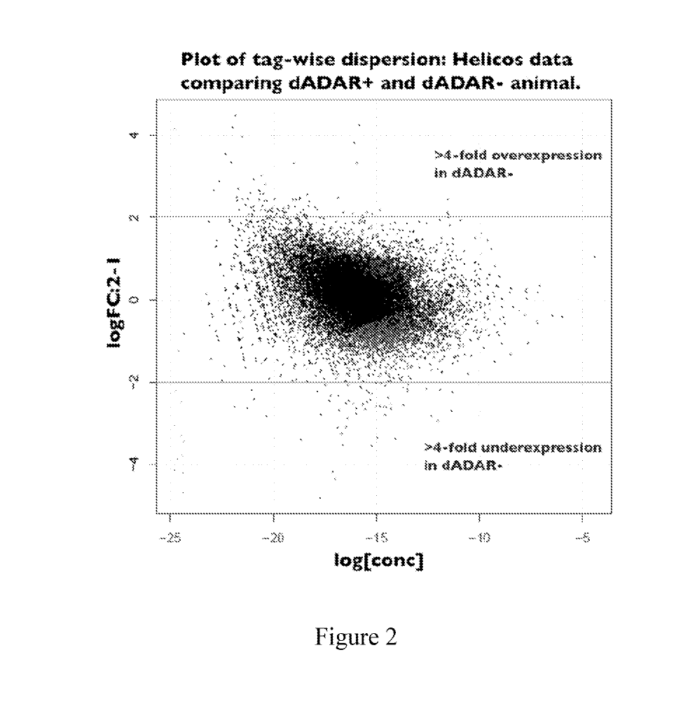

Deep sequencing using mRNAseq has revolutionized quantification of whole genome transcriptomes and largely replaces microarray technology by eliminating the possibility of cross-hybridization of probes to similar sequences (off-target effects), has very low background, and increases sensitivity for whole genome expression analyses (43). The method also offers increased output at decreased cost. In addition, mRNA-seq provides a report of non-coding RNAs, unannotated transcription events, and post-transcriptional processing events such as alternative splicing and RNA editing, all of possible importance in assessing the genome-wide response to disease-causing mutations in flies (44). As an example, a null mutant for the RNA editing enzyme, dADAR, was compared with a wild-type control (FIG. 2) using data obtained with a Helicos single-molecule sequencer. Perturbations of ADAR activity in humans has been implicated in ALS, epilepsy, suicidal depression and cancer. In flies, lack of ADAR leads to extreme neurological deficits and neurodegeneration. Over 80 million sequence tags were obtained from ADAR null and control animals. Sequenced reads mapped to the vast majority of the Drosophila genome (18,245 unique transcription units) and the range of detection was over 3 orders of magnitude. Moreover, altered expression was detected. Thus, it is expected that changes in transcriptional profiles in our dSOD mutants, as well as other backgrounds, can be detected.

### Example 4

### Phenotypic Characterization of Disease Progression

The genetically accurate model of ALS disclosed herein, can be used to elucidate not only the behavioral and electrophysiological changes, but also the cellular and molecular basis of disease progression. ALS-associated loss in motor neuron function and subsequent cell death has been attributed to defects in a number of processes from protein aggregation, increases is axonal membrane excitability, mitochondrial dysfunction, to changes in RNA metabolism and general metabolic homeostasis (49-55). This model system was used to explore the involvement of these processes in disease progression. Analyses of the most severe ALS-dSod1 mutant animals showed a progressive reduction in motility associated with an alteration in neural transmission culminating in death. The time of death varied between the different dSod1 mutations with <0.1% dSod1-G85R adults emerging from the pupal case (a feat requiring considerable muscle strength) to 33% emergence by the less severe dSod1-H71Y followed by death within 7-10 days of adulthood. The motility of the most severe dSod1-G85R mutant larvae was identical to the loxP control initially (in early 3rd instar larvae), but by late 3rd instar dSod1-G85R larvae displayed a significant reduction (40%) in motility. This decline in motor function continued and was manifested in the inability of dSod1-G85R adults to emerge from the pupal case (hence, they are referred to as pharates). These animals presented with an abnormal morphology of legs when dissected out of the pupal case, suggesting defects in musculature and/or innervation. Characterization of this progressive loss in motor function in detail during can be performed using immunohistochemical markers of muscle, as well as Drosophila lines expressing GFP-tagged muscle proteins (Mhc-mCD8-GFP) that allow visualization of muscles in living animals. Examination of the innervation of the muscles in controls and mutants is done by concomitant labeling of the MNs (CD8-RFP driven in MNs). This approach not only allows simultaneous detection of structural changes in muscles and their innervation, but to do so in living animals as time proceeds from the larva to the adult. Making use of transgenic lines with fluorescently marked muscles and MNs represents an advantage in assessing adults as the cuticle is a barrier to antibody penetration and monitoring the integrity of these tissues in adults that display a later onset is of interest, such as dSod1-H71Y that emerge but whose mobility declines resulting in a shortened lifespan.

Consistent with the reduced motility observed in dSod1-G85R late 3rd instar larvae, there was a concordant reduction in the spontaneous activity of MNs when recorded from larval muscles in preparations with the motor circuit intact (brain and ventral nerve cord (VNC) remain attached to the body wall muscles). In addition, immunohistochemical stainings of neuromuscular junctions (NMJs) showed less elaborate synaptic branching in dSod1-G85R larvae than in WT controls. Studies of spontaneous MN activity and NMJ structure in later stage mutant animals and in the other dSod1 alleles that exhibit later onset can be performed. These analyses, coupled with studies on muscle structure can provide a well-defined depiction of disease progression. While ALS paralysis results from a dramatic degeneration of MNs and atrophied muscles, it is unclear when and where the decline in function originates. It could begin in a specific set of cells or the loss of function could be a systemic effect. This can be addressed using the Drosophila model system disclosed herein. For example, directed expression of channel-rhodopsin (ChR2) to specific cells or neurons can be done, followed by excitation of those cells with a flash of light and recording of the response in the circuit output of a particular larval muscle. Driving UAS-ChR2 within different neurons (OK371Gal4 in motorneurons, ChaGal4 in cholinergic neurons, specific neural Gal4 lines from Janeila Farm collection) (55) can be done to address the integrity of the neural circuit and interrogate the function of individual neurons.

Gbb, the Drosophila orthologue of vertebrate BMPs 5, 6, 7, acted as a retrograde signal at the NMJ (56). Expression of Gbb in the muscle or in the MN expanded the NMJ, strengthening neurotransmission (57). In this example, expression of Gbb rescued the locomotor defect in dSod1-G85R and dSod1-H71Y mutant larvae, restored normal spontaneous MN activity to dSod1-G85R mutants, and increased G85R/H71Y eclosion (from 15% to 45% eclosion). This finding identifies a mechanistic handle to elucidate the molecular basis of ALS, and suggests that a potential therapeutic agent that may be used to decrease or possibly reverse disease progression. Taking advantage of cell-specific drivers (Ga14 lines), reagents to express or knock out Gbb signaling (UASgbb, UASgbbRNAi), as well as reagents to monitor activation of BMP signaling (i.e., anti-pMad, activation of target gene expression) dissection of the neural circuit can identify the precise points of Gbb action. Comparing properties of ALS mutants to wildtype and the Gbb-rescued animals improves the ability to identify critical pathways and processes at the root of motor function loss and lethality seen in ALS.

### Example 5

### Biomarkers of Disease

The response of cells to ALS-Sod1 mutations is poorly understood. A variety of processes have been shown to be defective in ALS patients including but not limited to: proteostasis defects, functional defects in mitochondria, management of oxidative stress, snRNA biogenesis, miRNA biogenesis, RNA splicing, and RNA editing (58-59). Given the preponderance of “RNA phenotypes” at the molecular level in diseases of motor neurons, detailed transcriptomic analyses can assess genome-wide responses of cells to mutant Sod1 expression and determine how cellular energy production may be compromised. In this example, the transcriptomes of ALS-dSod1 mutants at different stages of disease progression can be compared with loxP control and Gbb-rescued ALS-dSod1 mutants. RNAs from the ventral nerve cord (VNC), the origin of the MNs and the fat body (the major organ controlling metabolism) can be compared. This comparison can identify tissue-specific versus systemic changes. polyA+ RNA can be isolated from dissected tissues and prepared for Illumina sequencing. Samples can be barcoded to increase sample throughput per lane. In order to capture changes in gene expression, as well as to detect differences in RNA metabolism by identifying various RNA species, 100 bp paired end (PE) reads can be performed in the transcriptome analysis. The most severe allele, dSod1-G85R, its control (loxP), and the Gbb-rescued dSod1-G85R animals at four stages of disease progression (early L3, late L3, mid-pupal, and pharates or newly eclosed adults) can be tested. RNA-Seq data can be analyzed for gene expression changes as well as changes in RNA processing events of coding and noncoding RNAs. Follow up analysis of the severe dSod1-G85R allele with an examination of the transcriptomes from dSod1-H71 Y and the Gbb-rescued dSod1-H71Y with a focus on deciphering the genomic response to slower disease progression and its potential extension of not only lifespan but healthspan by Gbb rescue can be done. The same comparison of RNA sequences can be made of these genotypes over the four time points with an additional sample from “old” adults that have survived beyond 14 days post-eclosion.

Transcriptomic changes occurring in neurons in general (i.e. in the central brain) versus MNs in particular can be analyzed at two points in disease progression from moderate dSod1-H71 Y and severe dSod1-G85R animals and their Gbb-rescued counterparts. These analyses can be performed using tissue-specific tagging of RNA populations (TU-tagging) or INTACT RNAseq from neuronal nuclei (60-61).

Mitochondrial dysfunction and altered metabolism are associated with ALS. In this example, the bioenergetic state of the ALS mutants, disclosed herein, and their Gbb-rescued siblings can be tested, since in addition to Gbb's role in NMJ function, it has been shown that Gbb is critical for maintaining proper metabolic status (62). It can be determined if there is a progressive alteration in mitochondrial respiration, glycolysis and fatty acid oxidation concomitant with progressive loss in motor function in ALS-dSod1 mutants and the Gbb rescued animals using an XFe96 analyzer.

### REFERENCES

1—Polymenidou, M. and Cleveland, D. W. (2008) Motor neuron disease: The curious ways of ALS. Nature 454:284-285. PMID: 18633404
2—Maruyama, H., Morino, H., Ito, H., Izumi, Y., Kato, H., Watanabe, Y., Kinoshita, Y., Kamada, M., Nodera, H., Suzuki, H., Komure, O., Matsuura, S., Kobatake, K., Morimoto, N., Abe, K., Suzuki, N., Aoki, M., Kawata, A., Hirai, T., Kato, T., Ogasawara, K., Hirano, A., Takumi, T., Kusaka, H., Hagiwara K., Kaji, R., Kawakami, H. (2010) Mutations of optineurin in amyotrophic lateral sclerosis. Nature 465:223-226. PMID: 20428114
3—Lagier-Tourenne, C. and Cleveland, D. W. (2009) Rethinking ALS: the FUS about TDP-43. Cell 136:1001-1004. PMID: 19303844
4—Liu, J., Lillo, C., Jonsson, P. A., Vande Velde, C., Ward, C. M., Miller, T. M., Subramaniam, J. R., Rothstein, J. D., Marklund, S., Andersen, P. M., Brännström, T., Gredal, O., Wong, P. C., Williams, D. S. and Cleveland, D. W. (2004) Toxicity of familial ALS-linked SOD1 mutants from selective recruitment to spinal mitochondria. Neuron 43:5-17. PMID: 15233913
5—Turner B J, Atkin J D, Farg M A, Zang D W, Rembach A, Lopes E C, Patch J D, Hill A F, Cheema S. (2005) Impaired extracellular secretion of mutant superoxide dismutase 1 associates with neurotoxicity in familial amyotrophic lateral sclerosis. J Neurosci. 25(1):108-117. PMID: 15634772
6—Saxena S, Cabuy E, Caroni P. (2009) A role for motoneuron subtype-selective ER stress in disease manifestations of FALS mice. Nat Neurosci 12:627-636. PMID: 19330001
7—Nguyen, M. D., Larivière, R. C. and Julien, J. P. (2001) Deregulation of Cdk5 in a mouse model of ALS: toxicity alleviated by perikaryal neurofilament inclusions. Neuron. 30:135-147. PMID: 11343650
8—Duplan L, Bernard N, Casseron W, Dudley K, Thouvenot E, Honnorat J, Rogemond V, De Bovis B, Aebischer P, Marin P, Raoul C, Henderson C E, Pettmann B. (2010) Collapsin response mediator protein 4a (CRMP4a) is upregulated in motoneurons of mutant SOD1 mice and can trigger motoneuron axonal degeneration and cell death. J Neurosci. 30:785-796. PMID: 20071543
9—Elbasiouny S M, Amendola J, Durand J, Heckman C J. (2010) Evidence from computer simulations for alterations in the membrane biophysical properties and dendritic processing of synaptic inputs in mutant superoxide dismutase-1 motoneurons. J Neurosci. 30(16):5544-5558. PMID: 20410108
10—Guipponi M, Li Q X, Hyde L, Beissbarth T, Smyth G K, Masters C L, Scott H S. (2010) SAGE analysis of genes differentially expressed in presymptomatic TgSOD1G93A transgenic mice identified cellular processes involved in early stage of ALS pathology. J Mol Neurosci. 41:172-182. PMID: 19953340
11—Offen D, Barhum Y, Melamed E, Embacher N, Schindler C, Ransmayr G. (2009) Spinal cord mRNA profile in patients with ALS: comparison with transgenic mice expressing the human SOD-1 mutant. J Mol Neurosci. 38:85-93. PMID: 18651250
12—Lincecum J M, Vieira F G, Wang M Z, Thompson K, De Zutter G S, Kidd J, Moreno A, Sanchez R, Carrion I J, Levine B A, Al-Nakhala B M, Sullivan S M, Gill A, Perrin S. (2010) From transcriptome analysis to therapeutic anti-CD40L treatment in the SOD1 model of amyotrophic lateral sclerosis. Nat Genet. 42:392-399. PMID: 20348957
13—Rosen D R, Siddique T, Patterson D, Figlewicz D A, Sapp P, Hentati A, Donaldson D, Goto J, O'Regan J P, Deng H X, Rahmani Z, Krizus A, McKenna-Yasek D, Cayabyab A, Gaston S M, Berger R, Tanzi R E, Halperin, J J, Herzfeldt B, Van den Bergh R, Hung W Y, Bird T, Deng G, Mulder D W, Smyth C, Laing N G, Soriano E, Pericak-Vance M A, Haines J, Rouleau G A, Gusella J S, Horvitz H R, Brown R H. (1993) Mutations in Cu/Zn superoxide dismutase gene are associated with familial amyotrophic lateral sclerosis. Nature. 362:59-62. PMID: 8446170
14—Shaw B F, Lelie H L, Durazo A, Nersissian A M, Xu G, Chan P K, Gralla E B, Tiwari A, Hayward L J, Borchelt D R, Valentine J S, Whitelegge J P. (2008) Detergent-insoluble aggregates associated with amyotrophic lateral sclerosis in transgenic mice contain primarily full-length, unmodified superoxide dismutase-1. J Biol Chem. 283:8340-8350. PMID: 18192269
15—Gruzman A, Wood W L, Alpert E, Prasad M D, Miller R G, Rothstein J D, Bowser R, Hamilton R, Wood T D, Cleveland D W, Lingappa V R, Liu J. (2007) Common molecular signature in SOD1 for both sporadic and familial amyotrophic lateral sclerosis. Proc Natl Acad Sci 104(30):12524-12529. PMID: 17636119
16—Niwa J, Yamada S, Ishigaki S, Sone J, Takahashi M, Katsuno M, Tanaka F, Doyu M, Sobue G. (2007) Disulfide bond mediates aggregation, toxicity, and ubiquitylation of familial amyotrophic lateral sclerosis-linked mutant SOD1. J Biol Chem. 282:28087-28095. PMID: 17666395
17—Cozzolino M, Amori I, Pesaresi M G, Ferri A, Nencini M, Carrì M T. (2008) Cysteine 111 affects aggregation and cytotoxicity of mutant Cu,Zn-superoxide dismutase associated with familial amyotrophic lateral sclerosis. J Biol Chem. 283:866-874. PMID: 18006498
18—Banci L, Bertini I, Boca M, Girotto S, Martinelli M, Valentine J S, Vieru M. (2008) SOD1 and amyotrophic lateral sclerosis: mutations and oligomerization. PLoS One. 3:e1677. PMID: 18301754
19—Tan J M, Wong E S, Kirkpatrick D S, Pletnikova O, Ko H S, Tay S P, Ho M W, Troncoso J, Gygi S P, Lee M K, Dawson V L, Dawson T M, Lim K L. (2008) Lysine 63-linked ubiquitination promotes the formation and autophagic clearance of protein inclusions associated with neurodegenerative diseases. Hum Mol Genet. 17:431-439. PMID: 17981811
20—Niwa J, Ishigaki S, Hishikawa N, Yamamoto M, Doyu M, Murata S, Tanaka K, Taniguchi N, Sobue G. (2007) Dorfin ubiquitylates mutant SOD1 and prevents mutant SOD1-mediated neurotoxicity. J Biol Chem. 277:36793-36798. PMID: 12145308
21—Takeuchi H, Niwa J, Hishikawa N, Ishigaki S, Tanaka F, Doyu M, Sobue G. (2004) Dorfin prevents cell death by reducing mitochondrial localizing mutant superoxide dismutase 1 in a neuronal cell model of familial amyotrophic lateral sclerosis. J Neurochem. 89:64-72. PMID: 15030390
22—Sone J, Niwa J, Kawai K, Ishigaki S, Yamada S, Adachi H, Katsuno M, Tanaka F, Doyu M, Sobue G. (2010) Dorfin ameliorates phenotypes in a transgenic mouse model of amyotrophic lateral sclerosis. J Neurosci Res. 88:123-135. PMID: 19610091
23—Ishigaki S, Hishikawa N, Niwa J, Iemura S, Natsume T, Hori S, Kakizuka A, Tanaka K, Sobue G. (2004) Physical and functional interaction between Dorfin and Valosin-containing protein that are colocalized in ubiquitylated inclusions in neurodegenerative disorders. J Biol Chem. 279:51376-51385. PMID: 15456787
24—Wang J, Farr G W, Zeiss C J, Rodriguez-Gil D J, Wilson J H, Furtak K, Rutkowski D T, Kaufman R J, Ruse C I, Yates J R 3rd, Perrin S, Feany M B, Horwich A L. (2009) Progressive aggregation despite chaperone associations of a mutant SOD1-YFP in transgenic mice that develop ALS. Proc Natl Acad Sci USA. 106:1392-1397. PMID: 1917188
25—Bergemalm D, Forsberg K, Srivastava V, Graffmo K S, Andersen P M, Brännström T, Wingsle G, Marklund S L. (2010) Superoxide dismutase-1 and other proteins in inclusions from transgenic amyotrophic lateral sclerosis model mice. J Neurochem. 114:408-418 PMID: 20412382
26—Bronk P, Wenniger J J, Dawson-Scully K, Guo X, Hong S, Atwood H L, Zinsmaier K E. (2001) Drosophila Hsc70-4 is critical for neurotransmitter exocytosis in vivo. Neuron. 30:475-488. PMID: 11395008
27—Goldfarb S B, Kashlan O B, Watkins J N, Suaud L, Yan W, Kleyman T R, Rubenstein R C. (2006) Differential effects of Hsc70 and Hsp70 on the intracellular trafficking and functional expression of epithelial sodium channels. Proc Natl Acad Sci USA. 103:5817-5822. PMID: 16585520
28—Jaarsma D, Teuling E, Haasdijk E D, De Zeeuw C I, Hoogenraad C C. (2008) Neuron-specific expression of mutant superoxide dismutase is sufficient to induce amyotrophic lateral sclerosis in transgenic mice. J Neurosci. 28:2075-2088. PMID: 18305242
29—Mockett R J, Radyuk S N, Benes J J, On W C, Sohal R S. (2003) Phenotypic effects of familial amyotrophic lateral sclerosis mutant Sod alleles in transgenic Drosophila. Proc Natl Acad Sci USA. 100:301-306. PMID: 12502789
30—Watson M R, Lagow R D, Xu K, Zhang B, Bonini N M. (2008) A drosophila model for amyotrophic lateral sclerosis reveals motor neuron damage by human SOD1. J Biol Chem. 283:24972-49781. PMID: 18596033
31—Wang J, Farr G W, Hall D H, Li F, Furtak K, Dreier L, Horwich A L. (2009) An ALS-linked mutant SOD1 produces a locomotor defect associated with aggregation and synaptic dysfunction when expressed in neurons of Caenorhabditis elegans. PLoS Genet. 5:e1000350. PMID: 1916532
32—Maggert, K. A., Gong, W. J. and Golic, K. G. (2008) Methods for homologous recombination in Drosophila. Methods Mol Biol. 420:155-174.
33—Song, J. and Tanouye, M. A. (2008) From bench to drug: human seizure modeling using Drosophila. Prog Neurobiol. 84:182-191.
34—Jeibmann, A. and Paulus, W. (2009) Drosophila melanogaster as a Model Organism for Brain Diseases. Int J Mol Sci. 10:407-440.
35—Cauchi, R. J. and van den Heuvel M. (2006) The fly as a model for neurodegenerative diseases: is it worth the jump? Neurodegener Dis. 23:338-356.
36—Staber, C. J., Gell, S., Jepson, J. E. and Reenan, R. A. (2011) Perturbing A-to-I RNA Editing Using Genetics and Homologous Recombination. Methods Mol Biol 718:41-73. PMID: 21370041
37—O'Keefe, L. V., Smibert, P., Colella, A., Chataway, T. K., Saint, R. and Richards R. I. (2007) Know thy fly. Trends Genet. 23:238-242.
38—Weihl C C, Pestronk A, Kimonis V E. (2009) Valosin-containing protein disease: inclusion body myopathy with Paget's disease of the bone and fronto-temporal dementia. Neuromuscul Disord. 19:308-315. PMID: 19380227
39—Talbot K, Ansorge O. (2006) Recent advances in the genetics of amyotrophic lateral sclerosis and frontotemporal dementia: common pathways in neurodegenerative disease. Hum Mol Genet. 15:R182-187. PMID: 16987882
40—Gitcho M A, Strider J, Carter D, Taylor-Reinwald L, Forman M S, Goate A M, Cairns N J. (2009) VCP mutations causing frontotemporal lobar degeneration disrupt localization of TDP-43 and induce cell death. J Biol Chem. 284:12384-12398. PMID: 19237541
41—Ritson G P, Custer S K, Freibaum B D, Guinto J B, Geffel D, Moore J, Tang W, Winton M J, Neumann M, Trojanowski J Q, Lee V M, Forman M S, Taylor J P. (2010) TDP-43 mediates degeneration in a novel Drosophila model of disease caused by mutations in VCP/p97. J Neurosci. 30:7729-7739. PMID: 20519548
42—Noguchi M, Takata T, Kimura Y, Manno A, Murakami K, Koike M, Ohizumi H, Hori S, Kakizuka A. (2005) ATPase activity of p97/valosin-containing protein is regulated by oxidative modification of the evolutionally conserved cysteine 522 residue in Walker A motif. J Biol Chem. 280:41332-41341. PMID: 16234241
43—Mortazavi A, Williams B A, McCue K, Schaeffer L, Wold B. (2008) Mapping and quantifying mammalian transcriptomes by RNA-Seq. Nat Methods. 5:621-628. PMID: 18516045
44—Trapnell C, Williams B A, Pertea G, Mortazavi A, Kwan G, van Baren M J, Salzberg S L, Wold B J, Pachter L. (2010) Transcript assembly and quantification by RNA-Seq reveals unannotated transcripts and isoform switching during cell differentiation. Nat Biotechnol. 28:511-515. PMID: 20436464
45—Mockett R J, Bayne A C, Sohal B H, Sohal R S. (2002) Biochemical assay of superoxide dismutase activity in Drosophila. Methods Enzymol. 349:287-292. PMID: 11912918
46—Ruan H, Wu C F. (2008) Social interaction-mediated lifespan extension of Drosophila Cu/Zn superoxide dismutase mutants. Proc Natl Acad Sci USA. 105:7506-7510. PMID: 18508973
47—McManus C J, Coolon J D, Duff M O, Eipper-Mains J, Graveley B R, Wittkopp P J. (2010) Regulatory divergence in Drosophila revealed by mRNA-seq. Genome Res. 20:816-825. PMID: 20354124
48—Blumenstiel J P, Noll A C, Griffiths J A, Perera A G, Walton K N, Gilliland W D, Hawley R S, Staehling-Hampton K. (2009) Identification of EMS-induced mutations in Drosophila melanogaster by whole-genome sequencing. Genetics. 182:25-32. PMID: 19307605
49. Chen S, Sayana P, Zhang X, Le W. (2013) Genetics of amyotrophic lateral sclerosis: an update. Mol Neurodegener. 8:28. doi: 10.1186/1750-1326-8-28. PMID:23941283
50. Bosco D A, Morfini G, Karabacak N M, Song Y, Gros-Louis F et al., (2010) Wild-type and mutant SOD1 share an aberrant conformation and a common pathogenic pathway in ALS. Nat Neurosci. 13(11):1396-403. doi:10.1038/nn.2660. PMID:20953194
51. Kiskinis E, Sandoe J, Williams L A, Boulting G L, Moccia R et al., (2014) Pathways Disrupted in Human ALS Motor Neurons Identified through Genetic Correction of Mutant SOD1. Cell Stem Cell. S1934-5909(14)00098-8. doi: 10.1016/j.stem.2014.03.004. PMID:24704492
52. Lattante S, Rouleau G A, Kabashi E. (2013) TARDBP and FUS mutations associated with amyotrophic lateral sclerosis: summary and update. Hum Mutat. 34(6):812-26. doi: 10.1002/humu.22319. PMID: 23559573
53. Wainger B J, Kiskinis E, Mellin C, Wiskow O, Han S S et al., (2014) Intrinsic Membrane Hyperexcitability of Amyotrophic Lateral Sclerosis Patient-Derived Motor Neurons. Cell Rep. 2014 S2211-1247(14)00197-1. doi:10.1016/j.celrep.2014.03.019. PMID:24703839
54. Tan W, Pasinelli P, Trotti D. (2014) Role of mitochondria in mutant SOD1 linked amyotrophic lateral sclerosis. Biochim Biophys Acta. (14) 48-9. doi: 10.1016/PMID: 24568860
55. Pulver S R, Pashkovski S L, Hornstein N J, Garrity P A, Griffith L C. (2009) Temporal dynamics of neuronal activation by Channelrhodopsin-2 and TRPA1 determine behavioral output in Drosophila larvae. J Neurophysiol. 101(6):3075-88. doi: 10.1152/jn.00071.2009. PMID:19339465
56. McCabe B D, Marqués G, Haghighi A P, Fetter R D, Crotty M L et al., (2003) The BMP homolog Gbb provides a retrograde signal that regulates synaptic growth at the Drosophila neuromuscular junction. Neuron. 17; 39(2):241-54. PMID:12873382
57. James R E, Broihier H T. (2011) Crimpy inhibits the BMP homolog Gbb in motoneurons to enable proper growth control at the Drosophila neuromuscular junction. Development. 138(15):3273-86. doi:10.1242/dev.066142. PMID:21750037
58. Donnelly C J, Zhang P W, Pham J T, Heusler A R, Mistry N A., (2013) RNA toxicity from the ALS/FTD C9ORF72 expansion is mitigated by antisense intervention. Neuron. 80(2):415-28. doi: 10.1016/j.neuron.2013.10.015. PMID:24139042
59. Hideyama T, Yamashita T, Aizawa H, Tsuji S, Kakita A et al, (2012) Profound downregulation of the RNA editing enzyme ADAR2 in ALS spinal motor neurons. Neurobiol Dis. 45(3):1121-8. doi:10.1016/j.nbd.2011.12.033. PMID:22226999
60. Miller M R, Robinson K J, Cleary M D, Doe C Q. (2009) TU-tagging: cell type-specific RNA isolation from intact complex tissues. Nat Methods. 6(6):439-41. doi: 10.1038/nmeth.1329. PMID:19430475
61. Gilbert L. Henry, Fred P. Davis, Serge Picard, Sean R. Eddy (2012) Cell type-specific genomics of Drosophila neurons Nucleic Acids Res. 40(19): 9691-9704. doi: 10.1093/nar/gks671 PMCID: PMC3479168
62. Ballard S L, Jarolimova J, Wharton K A. (2010) Gbb/BMP signaling is required to maintain energy homeostasis in Drosophila. Dev Biol. 337(2):375-85. doi: 10.1016/j.ydbio.2009.11.011. PMID:19914231

### EQUIVALENTS

The foregoing written specification is considered to be sufficient to enable one ordinarily skilled in the art to practice the invention. The present invention is not to be limited in scope by examples provided, since the examples are intended as mere illustrations of one or more aspects of the invention. Other functionally equivalent embodiments are considered within the scope of the invention. Various modifications of the invention in addition to those shown and described herein will become apparent to those skilled in the art from the foregoing description. Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth or illustrated in the drawings. The invention is capable of other embodiments and of being practiced or of being carried out in various ways.

Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

All references, patents and patent applications that are recited in this application are incorporated by reference herein in their entirety.

What is claimed is:

1. A mutant Drosophila organism comprising a mutant dSOD1 gene, a humanized dSOD1 gene, or a partially humanized dSOD1 gene, wherein the mutant dSOD1 gene comprises at least one human-derived SOD1 mutation, and wherein the mutant dSOD1 gene, the humanized dSOD1 gene, or the partially humanized dSOD1 gene replaces at least one of the copies of the endogenous dSOD1 gene.

2. The mutant Drosophila organism of claim 1, wherein the human-derived SOD1 mutation is A4S, A4V, G37R, H48R, H71Y, G85R, R115G, D124G, G141E, G147R or C6S.

3. (canceled)

4. The mutant organism of claim 1, wherein the humanized dSOD1 gene, or the partially humanized dSOD1 gene further comprises at least one human-derived SOD1 mutation.

5. The mutant Drosophila organism of claim 4, wherein the human-derived SOD1 mutation is A4S, A4V, G37R, H48R, H71Y, G85R, R115G, D124G, G141E, G147R or C6S.

6. (canceled)

7. (canceled)

8. (canceled)

9. A method of making a dSOD1 mutant Drosophila organism, a humanized dSOD1 mutant Drosophila organism, or a partially humanized dSOD1 mutant Drosophila organism comprising the steps of:
(a) generating a nucleic acid targeting vector comprising a dSOD1 gene with at least one human-derived SOD1 mutation, a humanized dSOD1 gene, or a partially humanized dSOD1 gene;
(b) transforming a Drosophila organism with said targeting vector; and
(c) identifying mutants wherein the dSOD1 gene with at least one human-derived SOD1 mutation, the humanized dSOD1 gene, or the partially humanized dSOD1 gene replaces at least one of the copies of the endogenous dSOD1 gene.

10. The method of claim 9, wherein the human-derived SOD1 mutation is A4S, A4V, G37R, H48R, H71Y, G85R, R115G, D124G, G141E, G147R or C6S.

11. (canceled)

12. The method of claim 9, wherein the humanized dSOD1 gene, or the partially humanized dSOD1 gene comprises at least one human-derived SOD1 mutation.

13. The method of claim 12, wherein the human-derived SOD1 mutation is A4S, A4V, G37R, H48R, H71Y, G85R, R115G, D124G, G141E, G147R or C6S.

14. (canceled)

15. (canceled)

16. (canceled)

17. A method of identifying therapeutic ALS gene targets comprising the steps of:
(a) contacting the mutant organism of claim 4 with an effective amount of mutagen;
(b) generating progeny of said mutant organisms;
(c) identifying progeny wherein the phenotype of the mutant organism is suppressed or enhanced;
(d) sequencing the transcriptome of rescued progeny; and
(e) identifying the mutant genes.

18. A method of identifying a compound for treating an ALS patient in need of treatment comprising the steps of:
(a) contacting the mutant organism of claim 4 with a library of compounds; and
(b) identifying compounds that suppress the phenotype of the mutant organism.

19. A nucleic acid molecule comprising a dSOD1 gene having at least one human-derived SOD1 mutation wherein the nucleic acid further comprises nucleic acid sequences flanking said dSOD1 gene that integrate into the genome of a Drosophila organism and replace at least one endogenous dSOD1 gene.

20. The nucleic acid molecule of claim 19 wherein the dSOD1 gene is a humanized dSOD1 gene.

21. The nucleic acid molecule of claim 19 wherein the dSOD1 gene is a partially humanized dSOD1 gene.


SEQUENCE LISTING (3 sequences)

SEQ ID NO: 1 - DNA, 735 bp, Drosophila melanogaster
ccgcatgtatttctaagctgctctgctacggtcacaccatagaagatacctggaaagttctcaacttttttcgttttgataaattgattaattcattcgaaatggtggttaaagctgtctgcgtaattaacggcgatgccaagggcacggttttcttcgaacaggagagcagcggtacgcccgtgaaggtctccggtgaggtgtgcggcctggccaagggtctgcacggattccacgtgcacgagttcggtgacaacaccaatggctgcatgtcgtccggaccgcacttcaatccgtatggcaaggagcatggcgctcccgtcgacgagaatcgtcacctgggcgatctgggcaacattgaggccaccggcgactgccccaccaaggtcaacatcaccgactccaagattacgctcttcggcgccgacagcatcatcggacgcaccgttgtcgtgcacgccgatgccgatgatcttggccagggtggacacgagctgagcaagtcaacgggcaacgctggtgcccgcatcgggtgcggcgttattggcattgccaaggtctaagcgataatctattccgatgtcggccactgtgctgatctactctatttagcactacccactggagatatacaaacgatatacatacttctaaacataaatacatagcctgtggtctgttagttgatacgcaacctttgaggttcaataaattggtgttttgaaattgccccat

SEQ ID NO: 2 - protein, 153 aa, Drosophila melanogaster
MVVKAVCVINGDAKGTVFFEQESSGTPVKVSGEVCGLAKGLHGFHVHEFGDNTNGCMSSGPHFNPYGKEHGAPVDENRHLGDLGNIEATGDCPTKVNITDSKITLFGADSIIGRTVVVHADADDLGQGGHELSKSTGNAGARIGCGVIGIAKV

SEQ ID NO: 3 - protein, 154 aa, Homo sapiens [sequence omitted]